(19) United States
(12) Patent Application Publication
(10) Pub. No.: US 2007/0293780 A1
(43) Pub. Date: Dec. 20, 2007

(54) Capnography Apparatus

(76) Inventors: Aryeh Ben-Yosef, Mevasseret Zion (IL); Ephraim Carlebach, Ra'anana (IL); Uri Grach, Jerusalem (IL)

Correspondence Address: EITAN MEHULAL LAW GROUP, 116 JOHN ST,, SUITE 1201, NEW YORK, NY, 10038, US

(21) Appl. No.: 11/597,643

(22) PCT Filed:
(86) PCT No.: PCT/IL05/00547

Related U.S. Application Data
(60) Provisional application No. 60/575,174, filed on May 27, 2004.

(51) Int. Cl. A61B 5/08

(52) U.S. Cl. 600/532

(57) ABSTRACT

A switching solenoid valve for use in a fluid handling apparatus comprising two input ports for inputting fluid samples, and a third port for outputting fluid from one of the first and second ports. The valve switches one of the two input ports to the third port. The valve is such that the path between the ports which is normally closed when the solenoid is unactuated, has at least one of a significantly lower dead space, significantly less flow perturbations, and significantly lower total included volume between ports, than the path which is normally closed. Such a valve provides operational advantages especially for use in capnographic systems for analyzing exhaled breath.

### REFERENCE TO RELATED APPLICATIONS

Reference is made to U.S. Provisional Patent Application 60/575,174, filed May 27, 2004, entitled “MINIATURE SOLENOID VALVE”, the disclosure of which is hereby incorporated by reference and priority of which is hereby claimed pursuant to 37 CFR 1.78(a) (4) and (5)(i).

### FIELD OF THE INVENTION

The present invention relates to capnography generally and more particularly to capnographs employing solenoid valves.

### BACKGROUND OF THE INVENTION

The following U.S. Patent Documents are believed to represent the current state of the art:

U.S. Patent Nos. U.S. Pat. Nos. 5,085,402 and 6,024,114.

### SUMMARY OF THE INVENTION

The present invention seeks to provide capnography apparatus and a solenoid valve particularly advantageous for use therein.

There is thus provided in accordance with a preferred embodiment of the present invention a capnograph including a patient sample inlet, a reference sample inlet, a gas analysis chamber and a solenoid valve governing the supply of gas to the gas analysis chamber from the patient sample inlet and the reference sample inlet, the solenoid valve being operative for defining a normally-open passageway between the patient sample inlet and the gas analysis chamber and a normally-closed passageway between the reference sample inlet and the gas analysis chamber, the passageway between the patient sample inlet and the gas analysis chamber having significantly less dead space than the passageway between the reference sample inlet and the gas analysis chamber.

There is also provided in accordance with another preferred embodiment of the present invention a capnograph including a patient sample inlet, a reference sample inlet, a gas analysis chamber, a manifold and a solenoid valve governing the supply of gas to the gas analysis chamber from the patient sample inlet and the reference sample inlet, the solenoid valve being operative for defining a normally-open passageway between the patient sample inlet and the gas analysis chamber and a normally-closed passageway between the reference sample inlet and the gas analysis chamber, the manifold defining a socket for the solenoid valve and the passageways being defined in the manifold and jointly between the solenoid valve and the manifold at the socket.

There is further provided in accordance with yet another preferred embodiment of the present invention a capnograph including a patient sample inlet, a reference sample inlet, a gas analysis chamber and a solenoid valve governing the supply of gas to the gas analysis chamber from the patient sample inlet and the reference sample inlet, the solenoid valve being operative for defining a normally-open passageway between the patient sample inlet and the gas analysis chamber and a normally-closed passageway between the reference sample inlet and the gas analysis chamber, the capnograph being characterized in that it has a rise time which does not exceed 50 milliseconds at a flow rate of 50 ml/min.

More preferably, the rise time does not exceed 30 milliseconds at a flow rate of 50 ml/min. Most preferably, the rise time does not exceed 10 milliseconds at a flow rate of 50 ml/min.

There is yet further provided in accordance with still another preferred embodiment of the present invention a capnograph including a patient sample inlet, a reference sample inlet, a gas analysis chamber and a solenoid valve governing the supply of gas to the gas analysis chamber from the patient sample inlet and the reference sample inlet, the solenoid valve being operative for defining a normally-open passageway between the patient sample inlet and the gas analysis chamber and a normally-closed passageway between the reference sample inlet and the gas analysis chamber, the capnograph being characterized in that it has a rise time which does not exceed 10 milliseconds at a flow rate of 50 ml/min.

There is also provided in accordance with another preferred embodiment of the present invention a capnograph including a patient sample inlet, a reference sample inlet, a gas analysis chamber and a solenoid valve governing the supply of gas to the gas analysis chamber from the patient sample inlet and the reference sample inlet and including a magnet, the solenoid valve being operative for defining a normally-open passageway between the patient sample inlet and the gas analysis chamber and a normally-closed passageway between the reference sample inlet and the gas analysis chamber, wherein the passageway between the patient sample inlet and the gas analysis chamber is maintained open at least partially by a force applied by the magnet.

Preferably, the passageway between the patient sample inlet and the gas analysis chamber has significantly less dead space than the passageway between the reference sample inlet and the gas analysis chamber.

Preferably, the solenoid valve includes a partially hollow plunger. Additionally or alternatively, the solenoid valve includes a push valve. Alternatively or additionally, the solenoid valve includes a magnet operative to maintain the passageway between the patient sample inlet and the gas analysis chamber open irrespective of the orientation of the solenoid valve, when the solenoid valve is not actuated.

Preferably, the capnograph is characterized in that it has a rise time which does not exceed 50 milliseconds at a flow rate of 50 ml/min. More preferably, the rise time does not exceed 30 milliseconds at a flow rate of 50 ml/min. Most preferably, the rise time does not exceed 10 milliseconds at a flow rate of 50 ml/min.

There is further provided in accordance with yet another preferred embodiment of the present invention a gas analyzer including a patient sample inlet, a reference sample inlet, a gas analysis chamber and a solenoid valve governing the supply of gas to the gas analysis chamber from the patient sample inlet and the reference sample inlet, the solenoid valve being operative for defining a normally-open passageway between the patient sample inlet and the gas analysis chamber and a normally-closed passageway between the reference sample inlet and the gas analysis chamber, the passageway between the patient sample inlet and the gas analysis chamber having significantly less dead space than the passageway between the reference sample inlet and the gas analysis chamber.

There is even further provided in accordance with still another preferred embodiment of the present invention a gas analyzer including a patient sample inlet, a reference sample inlet, a gas analysis chamber, a manifold and a solenoid valve governing the supply of gas to the gas analysis chamber from the patient sample inlet and the reference sample inlet, the solenoid valve being operative for defining a normally-open passageway between the patient sample inlet and the gas analysis chamber and a normally-closed passageway between the reference sample inlet and the gas analysis chamber, the manifold defining a socket for the solenoid valve and the passageways being defined in the manifold and jointly between the solenoid valve and the manifold at the socket.

There is still further provided in accordance with another preferred embodiment of the present invention a gas analyzer including a patient sample inlet, a reference sample inlet, a gas analysis chamber and a solenoid valve governing the supply of gas to the gas analysis chamber from the patient sample inlet and the reference sample inlet, the solenoid valve being operative for defining a normally-open passageway between the patient sample inlet and the gas analysis chamber and a normally-closed passageway between the reference sample inlet and the gas analysis chamber, the gas analyzer being characterized in that it has a rise time which does not exceed 50 milliseconds at a flow rate of 50 ml/min.

More preferably, the rise time does not exceed 30 milliseconds at a flow rate of 50 ml/min. Most preferably, the rise time does not exceed 10 milliseconds at a flow rate of 50 ml/min.

There is also provided in accordance with yet another preferred embodiment of the present invention a gas analyzer including a patient sample inlet, a reference sample inlet, a gas analysis chamber and a solenoid valve governing the supply of gas to the gas analysis chamber from the patient sample inlet and the reference sample inlet, the solenoid valve being operative for defining a normally-open passageway between the patient sample inlet and the gas analysis chamber and a normally-closed passageway between the reference sample inlet and the gas analysis chamber, the gas analyzer being characterized in that it has a rise time which does not exceed 10 milliseconds at a flow rate of 50 ml/min.

There is further provided in accordance with still another preferred embodiment of the present invention a gas analyzer including a patient sample inlet, a reference sample inlet, a gas analysis chamber and a solenoid valve governing the supply of gas to the gas analysis chamber from the patient sample inlet and the reference sample inlet and including a magnet, the solenoid valve being operative for defining a normally-open passageway between the patient sample inlet and the gas analysis chamber and a normally-closed passageway between the reference sample inlet and the gas analysis chamber, wherein the passageway between the patient sample inlet and the gas analysis chamber is maintained open at least partially by a force applied by the magnet.

Preferably, the passageway between the patient sample inlet and the gas analysis chamber has significantly less dead space than the passageway between the reference sample inlet and the gas analysis chamber.

Preferably, the solenoid valve includes a partially hollow plunger. Additionally or alternatively, the solenoid valve includes a push valve. Alternatively or additionally, the solenoid valve includes a magnet operative to maintain the passageway between the patient sample inlet and the gas analysis chamber open irrespective of the orientation of the solenoid valve, when the solenoid valve is not actuated.

Preferably, the gas analyzer is characterized in that it has a rise time which does not exceed 50 milliseconds at a flow rate of 50 ml/min. More preferably, the rise time does not exceed 30 milliseconds at a flow rate of 50 ml/min. Most preferably, the rise time does not exceed 10 milliseconds at a flow rate of 50 ml/min.

### BRIEF DESCRIPTION OF THE DRAWINGS

The present invention will be understood and appreciated more fully from the following detailed description, taken in conjunction with the drawings in which:

FIG. 1 is a simplified pictorial illustration of a capnograph constructed and operative in accordance with a preferred embodiment of the present invention;

FIG. 2 is an exploded view illustration of part of the capnograph of FIG. 1, including a solenoid valve constructed and operative in accordance with a preferred embodiment of the present invention;

FIG. 3 is an assembled view illustration of the part of the capnograph shown in exploded view in FIG. 2;

FIGS. 4A and 4B illustrate gas flow through part of the capnograph of FIGS. 1-3 in respective patient sampling and reference sampling modes of operation; and

FIGS. 5A and 5B illustrate gas flow through part of a variation of the capnograph of FIGS. 1-3 in respective patient sampling and reference sampling modes of operation.

### DETAILED DESCRIPTION OF PREFERRED EMBODIMENTS

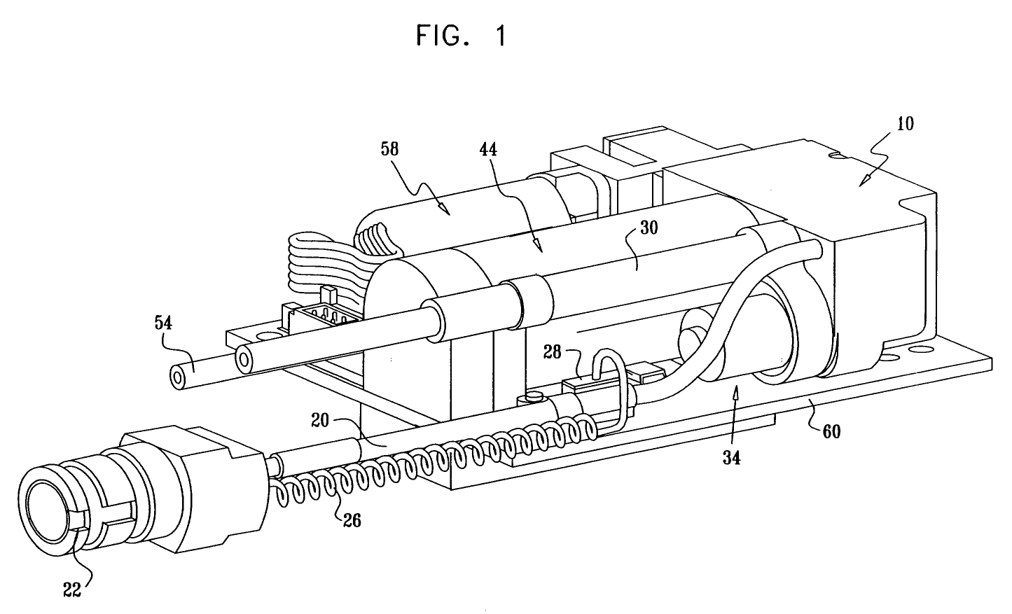

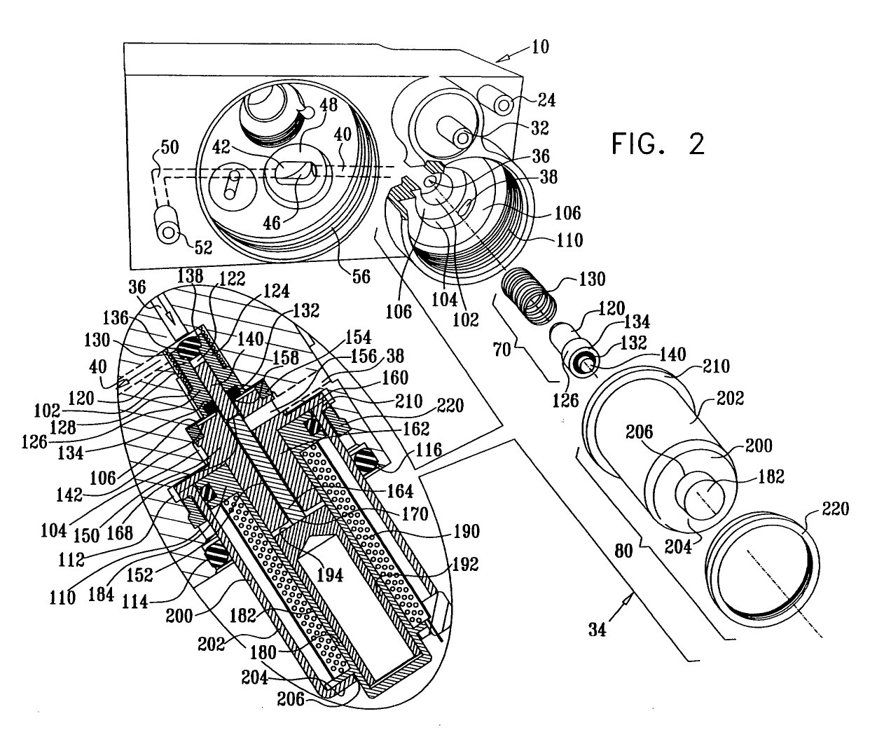

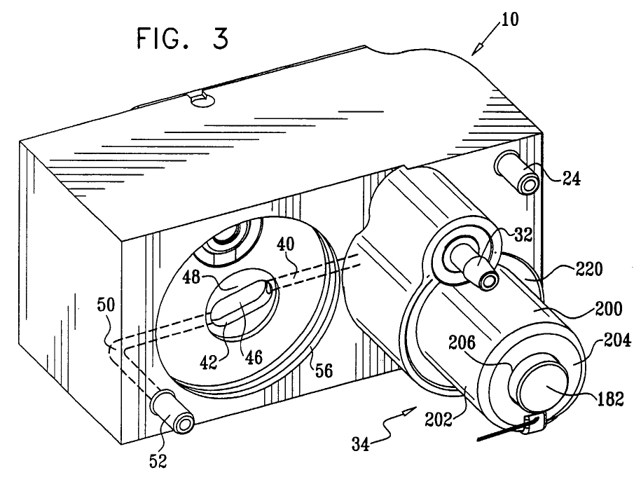

Reference is now made to FIG. 1, which is a simplified pictorial illustration of a capnograph constructed and operative in accordance with a preferred embodiment of the present invention, to FIG. 2, which is an exploded view illustration of part of the capnograph of FIG. 1, including a solenoid valve constructed and operative in accordance with a preferred embodiment of the present invention, and to FIG. 3, which is an assembled view illustration of the part of the capnograph shown in FIG. 2.

As seen most clearly in FIG. 1, the capnograph comprises a main housing element 10. A patient breath input tube 20, having an input connector 22, which is connectable to a source of patient breath, is attached to a patient gas input port 24 (FIGS. 2 & 3) formed in main housing element 10. A spiraled cable 26 typically is operative to transmit data in electronic form between input connector 22 and a microprocessor 28 which governs the operation of the capnograph. A reference gas input tube 30 is attached to a reference gas input port 32 formed in the main housing element 10.

Threadably mounted onto main housing element 10 is a solenoid valve assembly 34, communicating with a patient sample input bore 36 and a reference input bore 38 formed in main housing element 10 and connected, via additional bores (not shown) formed in the main housing element 10, to the patient gas input port 24 and the reference gas input port 32 respectively.

Gas entering the capnograph from either of patient breath input tube 20 and reference gas input tube 30 passes through the solenoid valve assembly 34 and thence via a gas supply bore 40 to a gas analysis chamber 42 formed within main housing element 10. In the gas analysis chamber 42 the gas is analyzed using an infrared lamp assembly 44 emitting infrared light which passes through a window portion 46 formed in a wall 48 of gas analysis chamber 42. Gas leaves the gas analysis chamber 42 via a bore 50, formed in main housing element 10, leading to a gas output port 52 which is connected to a gas output tube 54.

It is appreciated that the patient sample input bore 36, reference input bore 38 and gas supply bore 40, as well as other bores referred to herein, may extend in various planes of the main housing element 10, and typically do not all extend in a single plane of the main housing element 10 as depicted for the sake of clarity, in the drawings.

The solenoid valve assembly 34 governs the supply of gas to gas analysis chamber 42 from the patient sample input bore 36 and the reference input bore 38.

Infrared lamp assembly 44 preferably includes an infrared lamp (not shown) which is threadably connected to a threaded bore 56 formed in the main housing element 10, and receives electrical power from a power source 58. Typically, main housing element 10, infrared lamp assembly 44 and power source 58 are mounted onto a base element 60.

As seen with particular clarity in FIG. 2, the solenoid valve assembly 34 includes a valve subassembly 70 and a solenoid subassembly 80.

Main housing element 10 is configured to accommodate the valve subassembly 70 and the solenoid subassembly 80 and includes a generally cylindrical bore 102 which is in fluid flow communication with patient sample input bore 36 and gas supply bore 40.

Rearward of cylindrical bore 102, in the sense of FIG. 2, there is formed a generally cylindrical bore 104, which has a larger cross-section than that of cylindrical bore 102, and a shoulder 106 is defined between bores 102 and 104. Cylindrical bore 104 is in fluid flow communication with reference input bore 38.

Rearward of bore 104 in the sense of FIG. 2, there is formed a generally threaded cylindrical bore 110, having a cross-section which is larger than that of bore 104. Forward and rearward ends of bore 110, designated by reference numerals 112 and 114 respectively, have somewhat larger cross sections than the remainder of bore 110. Bores 104 and 110 accommodate solenoid subassembly 80, and a sealing ring 116 is located at end 114.

Valve subassembly 70 includes a body portion 120 which is loosely and slidingly accommodated within cylindrical bore 102 of main housing element 10. Body portion 120 is formed with a bore 122 extending axially therethrough, and includes a first generally cylindrical portion 124 having a first cross-section, and a second generally cylindrical portion 126 having a second cross section which is generally larger than that of cylindrical portion 124.

A shoulder 128 is defined between cylindrical portions 124 and 126 and defines a seat for a compression spring 130, disposed about cylindrical portion 124.

A seal 132 is located in a recess 134 formed at a rearward facing surface of cylindrical portion 126.

Disposed at a forward end of bore 122 is an additional bore 136 which has a larger cross section than that of bore 122. A flexible elastomeric sealing element 138 is sealingly seated within bore 136 and extends rearwardly into a forward portion of bore 122.

A shaft 140 is fixedly seated within bore 122 and is axially rearwardly spaced from elastomeric sealing element 138. Shaft 140 extends rearwardly through seal 132 and out of bore 122. Alternatively, shaft 140 may be integrally formed with body portion 120.

Solenoid subassembly 80 includes a forward element 150, a forward portion of which is seated within cylindrical bore 104 of main housing element 10. Forward element 150 is formed with a bore 152 extending axially therethrough, and includes a forwardly facing generally cylindrical portion 154. Cylindrical portion 154 is formed with a transverse bore 156, which is arranged to be in fluid flow communication with reference input bore 38 formed in main housing element 10.

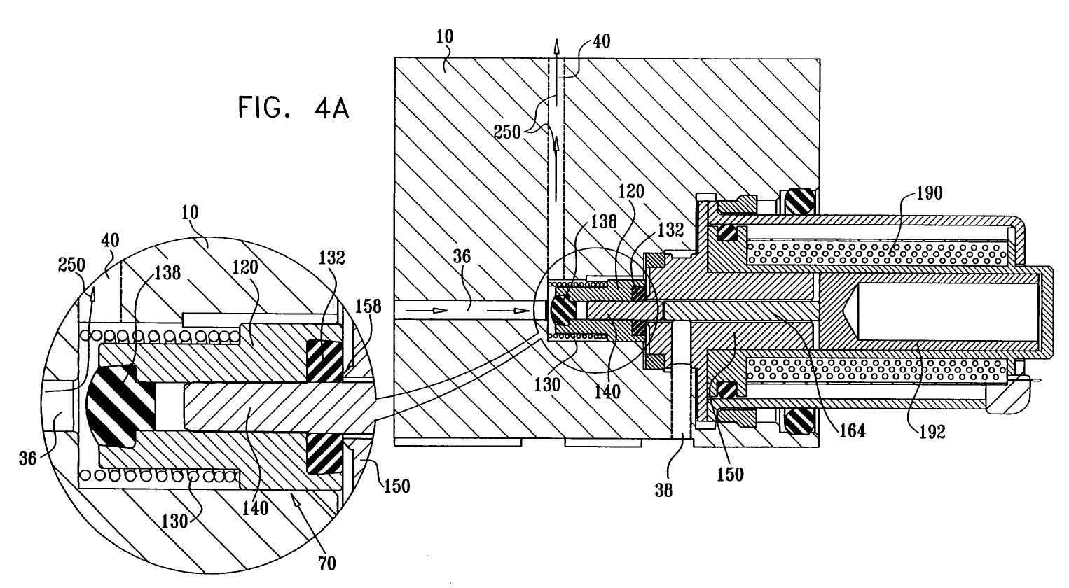

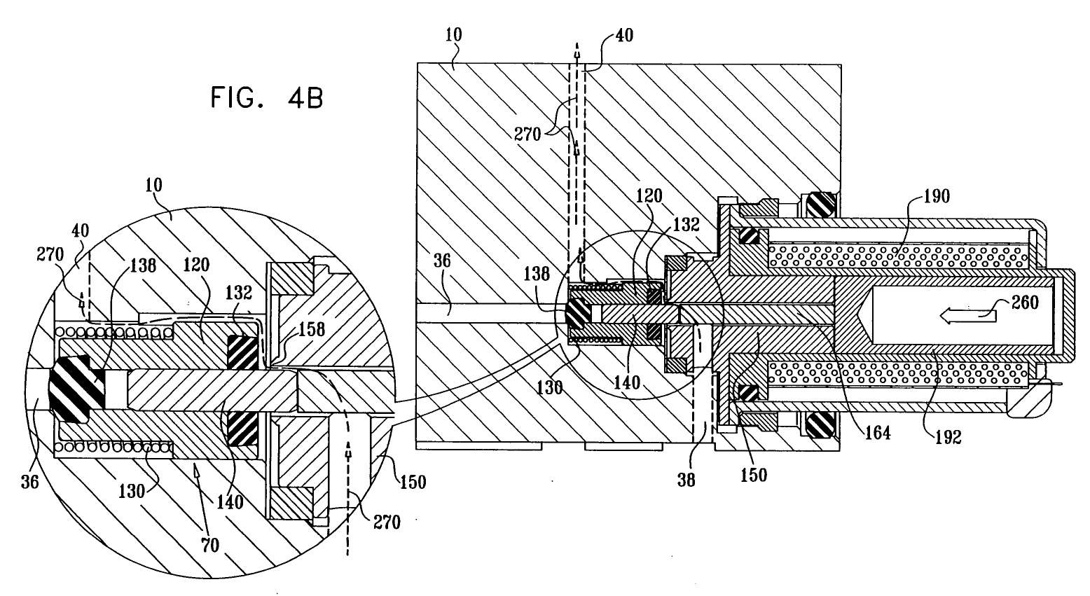

At a forward end thereof, cylindrical portion 154 includes a ring shaped protrusion 158, which is best seen in FIGS. 4A and 4B, described hereinbelow. Ring shaped protrusion 158 is adapted to sealingly engage seal 132.

Forward element 150 also includes, integrally formed with cylindrical portion 154 and rearwardly thereof, a disc portion 160, rearwardly of which there is formed a generally cylindrical portion 162.

At a forward end thereof, bore 152 slidingly accommodates a rearwardly facing end of shaft 140 of valve subassembly 70. A shaft 164, having a forward facing surface 168 and a rearward facing surface 170, is slidingly disposed within bore 152 rearwardly of shaft 140. Forward facing surface 168 of shaft 164 engages a rearward facing surface of shaft 140, and rearward-facing surface 170 of shaft 164 extends rearwardly and axially outwardly of forward element 150.

Solenoid subassembly 80 additionally includes a tubular coil support element 180 having a tubular portion 182. At a forward end thereof, tubular coil support element 180 includes a flange portion 184. Tubular coil support element 180 is disposed about cylindrical portion 154 of forward element 150 and extends rearwardly thereof. A solenoid 190 is wound about tubular portion 182 of tubular coil support element 180.

A plunger 192, which is preferably partially hollow and which defines a forward facing surface 194, is slidingly disposed within tubular portion 182 of tubular coil support element 180. Forward facing surface 194 of plunger 192 engages rearward facing surface 170 of shaft 164.

A solenoid housing 200 includes a generally cylindrical tubular portion 202 which terminates at a rearward end thereof in a wall portion 204. Wall portion 204 is formed with a generally circular aperture 206 which accommodates a rearward facing portion of tubular portion 182. Solenoid housing 200 defines at a forward end thereof a flange portion 210 which abuts against disk portion 160.

A nut 220, which surrounds solenoid housing 200, is threadably seated within bore 110 of main housing element 10, thus retaining valve subassembly 70 and solenoid subassembly 80 therein.

Reference is now made to FIGS. 4A and 4B, which illustrate gas flow through part of the capnograph of FIGS. 1-3 in respective patient sampling and reference sampling modes of operation.

FIG. 4A illustrates a patient sampling mode of operation, during which current does not flow through solenoid 190, and valve subassembly 70 is in a rearward, normally open position. In this normally open position, a fluid flow passageway designated by arrows 250 extending from patient sample input bore 36 of main housing element 10 to gas supply bore 40 is open. In this mode of operation, seal 132 sealingly engages protrusion 158 of forward element 150, thus minimizing the dead space in the fluid flow passageway. The valve subassembly 70 is maintained in this open position by the force of compression spring 130 and does not require electrical power.

In the patient sampling mode of operation, as shown in FIG. 4A, a gas sample which is supplied to the solenoid valve assembly 34 flows freely from patient sample input bore 36 to gas supply bore 40, with little or no interference. It is a particular feature of the present invention that in the patient sampling mode of operation, there is very little dead-space in the passageway designated by arrows 250, thus reducing distortion of the waveform reaching the gas analysis chamber 42 and causing the rise-time thereof to be relatively low, preferably less than 50 milliseconds, more preferably less than 30 milliseconds and most preferably not exceeding 10 milliseconds.

In the patient sampling mode of operation, a passageway defined between reference input bore 38 and gas supply bore 40 is normally closed, and the passageway designated by arrows 250 has significantly less dead space than the passageway defined between reference input bore 38 and gas supply bore 40.

FIG. 4B illustrates a reference sampling mode of operation, during which a current flows through solenoid 190, thereby pushing plunger 192 axially forward against the force applied by compression spring 130, in a direction indicated by an arrow 260. Forward motion of plunger 192 results in respective forward motion of shaft 164, which causes forward motion of shaft 140 and of body portion 120, resulting in elastomeric sealing element 138 sealingly engaging patient sample input bore 36.

In this closed position, a fluid flow passageway, designated by arrows 270, extending from reference input bore 38 of main housing element 10 to gas supply bore 40 is open. In this mode of operation, seal 132 does not engage protrusion 158 of forward element 150. The valve subassembly 70 is maintained in this closed position by the force of the magnetic field created by passing a current through solenoid 190.

In the reference sampling mode of operation, as shown in FIG. 4B, a gas sample which is supplied to the solenoid valve assembly 34 flows generally freely from reference input bore 38 to gas supply bore 40. Although there is dead space surrounding the fluid passageway indicated by arrows 270, this dead-space does not affect the accuracy of the analysis of the reference gas, as the waveform of the reference gas is of no importance in the testing.

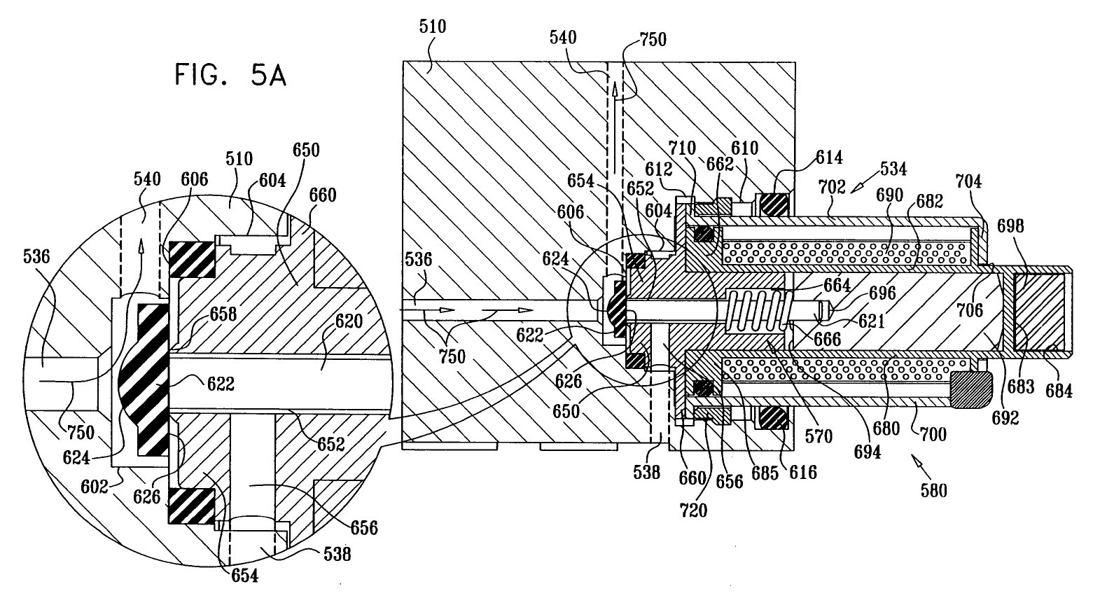

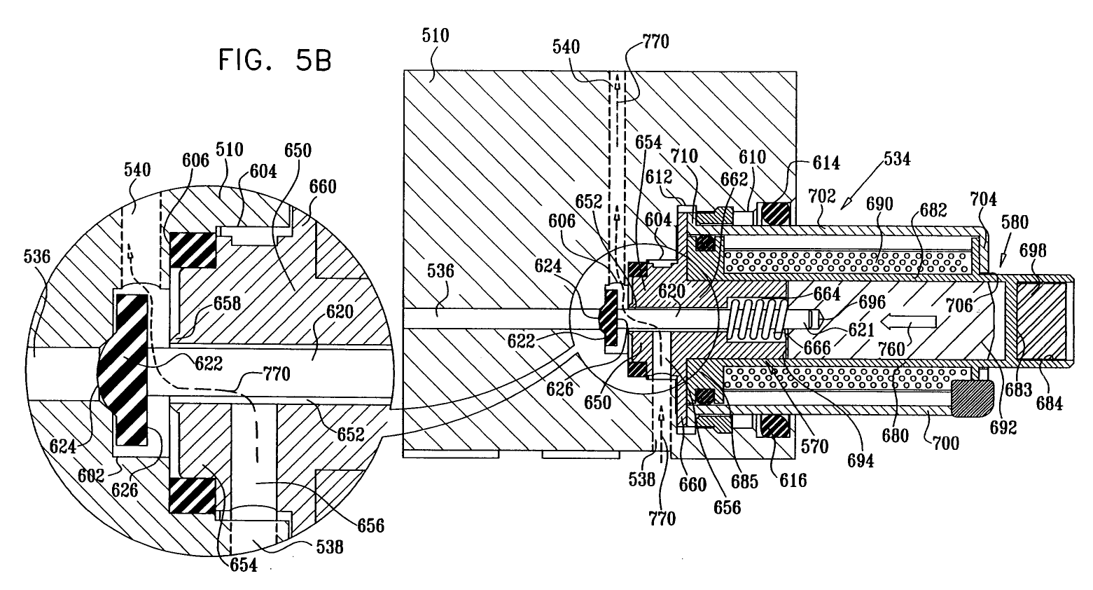

Reference is now made to FIGS. 5A and 5B, which illustrate gas flow through part of a variation of the capnograph of FIGS. 1-3 in respective patient sampling and reference sampling modes of operation.

As seen in FIGS. 5A and 5B, the capnograph comprises a main housing element 510. Threadably mounted onto main housing element 510 is a solenoid valve assembly 534, communicating with a patient sample input bore 536 and a reference input bore 538 formed in main housing element 510 and connected, via additional bores (not shown) formed in the main housing element 510, to a patient gas input port (not shown) and a reference gas input port (not shown) respectively.

Gas entering the capnograph from either of a patient breath input tube and a reference gas input tube passes through the solenoid valve assembly 534 and thence via a gas supply bore 540 to a gas analysis chamber (not shown) formed within main housing element 510.

In a similar manner to that described hereinabove with reference to FIG. 1, the gas is analyzed in a gas analysis chamber by infrared light emitted from an infrared lamp assembly. Gas leaves the gas analysis chamber via a bore formed in main housing element 510 and a gas output port which is connected to a gas output tube.

It is appreciated that the patient sample input bore 536, reference input bore 538 and gas supply bore 540 as well as other bores referred to herein may extend in various planes of the main housing element 510, and typically do not all extend in a single plane of the main housing element 510 as depicted for the sake of clarity, in FIGS. 5A and 5B.

The solenoid valve assembly 534 includes a valve subassembly 570 and a solenoid subassembly 580. Main housing element 510 is configured to accommodate the valve subassembly 570 and the solenoid subassembly 580 and includes a generally cylindrical bore 602 which is in fluid flow communication with patient sample input bore 536 and gas supply bore 540. Rearward of cylindrical bore 602, in the sense of FIG. 5A, there is formed a generally cylindrical bore 604, which has a larger cross-section than that of cylindrical bore 602, and a shoulder 606 is defined between bores 602 and 604. Cylindrical bore 604 is in fluid flow communication with reference input bore 538.

Rearward of bore 604, there is formed a generally threaded cylindrical bore 610, having a cross-section which is larger than that of bore 604. Forward and rearward ends of bore 610, designated by reference numerals 612 and 614, respectively have somewhat larger cross sections than the remainder of bore 610. A sealing ring 616 is located at end 614.

Valve subassembly 570 includes a shaft portion 620 defining a rearward facing end portion 621 and having an elastomeric sealing element 622 mounted on a forward end thereof. Elastomeric sealing element defines a forward facing surface 624 and a rearward facing surface 626, and is loosely and slidingly accommodated within cylindrical bore 602.

Solenoid subassembly 580 includes a forward element 650, a forward portion of which is seated within cylindrical bore 604 of main housing element 510. Forward element 650 is formed with a bore 652 extending axially therethrough, and includes a forwardly facing generally cylindrical portion 654. Cylindrical portion 654 is formed with a transverse bore 656, which is arranged to be in fluid flow communication with reference input bore 538 formed in main housing element 510.

At a forward end thereof, cylindrical portion 654 includes a ring shaped protrusion 658, which is best seen in the enlarged portions of FIGS. 5A and 5B. Ring shaped protrusion 658 is adapted to sealingly engage rearward facing surface 626 of elastomeric sealing element 622.

Forward element 650 also includes, integrally formed with cylindrical portion 654 and rearwardly thereof, a disc portion 660, rearwardly of which there is formed a generally cylindrical portion 662. A recess 664 is formed at a rearward facing surface of cylindrical portion 662, defines a spring seat for a compression spring 666, which is disposed about shaft 620.

Bore 652 loosely and slidingly accommodates shaft 620 of valve subassembly 570.

Solenoid subassembly 580 additionally includes a tubular coil support element 680 having a tubular portion 682 terminating at a wall portion 683, rearward of which there is formed a cylindrical portion 684. At a forward end thereof, tubular coil support element 680 includes a flange portion 685. Tubular coil support element 680 is disposed about cylindrical portion 654 of forward element 650 and extends rearwardly thereof. A solenoid 690 is wound about tubular portion 682 of tubular coil support element 680.

A plunger 692, which defines a forward surface 694, is slidingly disposed within tubular portion 682 of tubular coil support element 680. Forward surface 694 of plunger 692 defines a rear spring seat for compression spring 666. A bore 696, formed in a forward facing portion of plunger 692, fixedly accommodates rearward facing end 621 of shaft 620. Preferably a magnet 698 is seated within cylindrical portion 684 against wall portion 683, thus maintaining plunger 692 in its rear position when the solenoid 690 is not actuated.

A solenoid housing 700 includes a generally cylindrical tubular portion 702 which terminates at a rearward end thereof in a wall portion 704. Wall portion 704 is formed with a generally circular aperture 706 which accommodates a rearward facing portion of tubular portion 682. Solenoid housing 700 defines at a forward end thereof a flange portion 710 which abuts against disk portion 660.

A nut 720, which surrounds solenoid housing 700, is threadably seated within bore 610 of main housing element 510, thus retaining valve subassembly 570 and solenoid subassembly 580 therein.

An essential difference between the embodiment of FIGS. 1-4B and that of FIGS. 5A and 5B is that the compression spring is placed in a more rearward position in the solenoid valve subassembly 534, resulting in a further reduction of dead space in the system.

FIG. 5A illustrates a patient sampling mode of operation, during which current does not flow through solenoid 690, and valve subassembly 570 is in a rearward, normally open position. In this normally open position, a fluid flow passageway designated by arrows 750 extending from sample input bore 536 of main housing element 510 to gas supply bore 540 is open. In this mode of operation, ring shaped protrusion 658 of forward element 650 sealingly engages rearward facing surface 626 of elastomeric sealing element 622, thus minimizing the dead space in the fluid flow passageway. The valve subassembly 570 is maintained in this open position by the force of compression spring 666 and does not require electrical power. Additionally, magnet 698 maintains plunger 692 in its rear position, thus ensuring that the valve subassembly 570 remains in its open position irrespective of its orientation, when solenoid 690 is not actuated.

In the patient sampling mode of operation, as shown in FIG. 5A, a gas sample which is supplied to the solenoid valve assembly 534 flows freely from sample input bore 536 to gas supply bore 540, with little or no interference. It is a particular feature of the present invention that in the patient sampling mode of operation, there is very little dead-space along or in communication with the passageway designated by arrows 750, thus ensuring that unnecessary distortion of the waveform reaching the gas analysis chamber is avoided and the rise-time is relatively low, preferably less than 50 milliseconds, more preferably less than 30 milliseconds and most preferably not exceeding 10 milliseconds.

In the patient sampling mode of operation, a passageway defined between reference input bore 538 and gas supply bore 540 is normally closed, and the passageway designated by arrows 750 has significantly less dead space than the passageway defined between reference input bore 538 and gas supply bore 540.

FIG. 5B illustrates a reference sampling mode of operation, during which a current flows through solenoid 690. The force of the magnetic field formed by the current flowing through the solenoid 690 initally enables the release of plunger 692 from magnet 698, and thereafter enables motion of plunger 692 axially forward against the force applied by compression spring 666, in a direction indicated by an arrow 760. It is a particular feature of the present invention that the force required to displace the plunger 692 away from magnet 698 is equal to or less than the force required to push the plunger forward against the force applied by compression spring 666. Forward motion of plunger 692 results in respective forward motion of shaft 620 and elastomeric sealing element 622 and in sealing engagement between forward facing surface 624 of elastomeric sealing element 622 and sample input bore 536.

In this closed position, a fluid flow passageway designated by arrows 770 extending from reference input bore 538 of main housing element 510 to gas supply bore 540 is open. In this mode of operation, rearward facing surface 626 of elastomeric sealing element 622 does not engage protrusion 658 of forward element 650. The valve subassembly 570 is maintained in this closed position by the force of the magnetic field created by passing a current through solenoid 690.

In the reference sampling mode of operation, as shown in FIG. 5B, a gas sample which is supplied to the solenoid valve assembly 534 flows generally freely from reference input bore 538 to gas supply bore 540. Though there is dead space surrounding the fluid passageway indicated by arrows 770, this dead-space does not affect the accuracy of the analysis of the reference gas, as the waveform of the reference gas is of no importance in the testing.

It is appreciated by persons skilled in the art that the present invention is not limited by what has been particularly shown and described hereinabove. Rather the scope of the present invention includes both combinations and subcombinations of various features described hereinabove as well as variations and modifications thereto which would occur to a person of skill in the art upon reading the above description and which are not in the prior art.

1. A capnograph comprising: a patient sample inlet; a reference sample inlet; a gas analysis, chamber; and a solenoid valve governing the supply of gas to said gas analysis chamber from said patient sample inlet and said reference sample inlet, said solenoid valve being operative for defining a normally-open passageway between said patient sample inlet and said gas analysis chamber and a normally-closed passageway between said reference sample inlet and said gas analysis chamber, said passageway between said patient sample inlet and said gas analysis chamber having significantly less dead space than said passageway between said reference sample inlet and said gas analysis chamber.

2. A capnograph comprising: a patient sample inlet; a reference sample inlet; a gas analysis chamber; a manifold; and a solenoid valve governing the supply of gas to said gas analysis chamber from said patient sample inlet and said reference sample inlet, said solenoid valve being operative for defining a normally-open passageway between said patient sample inlet and said gas analysis chamber and a normally-closed passageway between said reference sample inlet and said gas analysis chamber, said manifold defining a socket for said solenoid valve and said passageways being defined in said manifold and jointly between said solenoid valve and said manifold at said socket.

3. A capnograph comprising: a patient sample inlet; a reference sample inlet; a gas analysis chamber; and a solenoid valve governing the supply of gas to said gas analysis chamber from said patient sample inlet and said reference sample inlet, said solenoid valve being operative for defining a normally-open passageway between said patient sample inlet and said gas analysis chamber and a normally-closed passageway between said reference sample inlet and said gas analysis chamber, said capnograph being characterized in that it has a rise time which does not exceed 50 milliseconds at a flow rate of 50 ml/min.

4. A capnograph according to claim 3, and wherein said rise time does not exceed 30 milliseconds at a flow rate of 50 ml/min.

5. A capnograph according to claim 3, and wherein said rise time does not exceed 10 milliseconds at a flow rate of 50 ml/min.

6. A capnograph comprising: a patient sample inlet; a reference sample inlet; a gas analysis chamber; and a solenoid valve governing the supply of gas to said gas analysis chamber from said patient sample inlet and said reference sample inlet, said solenoid valve being operative for defining a normally-open passageway between said patient sample inlet and said gas analysis chamber and a normally-closed passageway between said reference sample inlet and said gas analysis chamber, said capnograph being characterized in that it has a rise time which does not exceed 10 milliseconds at a flow rate of 50 ml/min.

7. A capnograph comprising: a patient sample inlet; a reference sample inlet; a gas analysis chamber; and a solenoid valve governing the supply of gas to said gas analysis chamber from said patient sample inlet and said reference sample inlet and comprising a magnet, said solenoid valve being operative for defining a normally-open passageway between said patient sample inlet and said gas analysis chamber and a normally-closed passageway between said reference sample inlet and said gas analysis chamber, wherein said passageway between said patient sample inlet and said gas analysis chamber is maintained open at least partially by a force applied by said magnet.

8. A capnograph according to claim 2 and wherein said passageway between said patient sample inlet and said gas analysis chamber has significantly less dead space than said passageway between said reference sample inlet and said gas analysis chamber

9. A capnograph according to claim 1 and wherein said solenoid valve comprises a partially hollow plunger.

10. A capnograph according to claim 1 and wherein said solenoid valve comprises a push valve.

11. A capnograph according to claim 1 and wherein said solenoid valve comprises a magnet operative to maintain said passageway between said patient sample inlet and said gas analysis chamber open irrespective of the orientation of said solenoid valve, when said solenoid valve is not actuated.

12. A capnograph according to claim 1, said capnograph being characterized in that it has a rise time which does not exceed 50 milliseconds at a flow rate of 50 ml/min.

13. A capnograph according to claim 12, and wherein said rise time does not exceed 30 milliseconds at a flow rate of 50 ml/min.

14. A capnograph according to claim 12, and wherein said rise time does not exceed 10 milliseconds at a flow rate of 50 ml/min.

15. A gas analyzer comprising: a patient sample inlet; a reference sample inlet; a gas analysis chamber; and a solenoid valve governing the supply of gas to said gas analysis chamber from said patient sample inlet and said reference sample inlet, said solenoid valve being operative for defining a normally-open passageway between said patient sample inlet and said gas analysis chamber and a normally-closed passageway between said reference sample inlet and said gas analysis chamber, said passageway between said patient sample inlet and said gas analysis chamber having significantly less dead space than said passageway between said reference sample inlet and said gas analysis chamber.

16. A gas analyzer comprising: a patient sample inlet; a reference sample inlet; a gas analysis chamber; a manifold; and a solenoid valve governing the supply of gas to said gas analysis chamber from said patient sample inlet and said reference sample inlet, said solenoid valve being operative for defining a normally-open passageway between said patient sample inlet and said gas analysis chamber and a normally-closed passageway between said reference sample inlet and said gas analysis chamber, said manifold defining a socket for said solenoid valve and said passageways being defined in said manifold and jointly between said solenoid valve and said manifold at said socket.

17. A gas analyzer comprising: a patient sample inlet; a reference sample inlet; a gas analysis chamber; and a solenoid valve governing the supply of gas to said gas analysis chamber from said patient sample inlet and said reference sample inlet, said solenoid valve being operative for defining a normally-open passageway between said patient sample inlet and said gas analysis chamber and a normally-closed passageway between said reference sample inlet and said gas analysis chamber, said gas analyzer being characterized in that it has a rise time which does not exceed 50 milliseconds at a flow rate of 50 ml/min.

18. A gas analyzer according to claim 17, and wherein said rise time does not exceed 30 milliseconds at a flow rate of 50 ml/min.

19. A gas analyzer according to claim 17, and wherein said rise time does not exceed 10 milliseconds at a flow rate of 50 ml/min.

20. A gas analyzer comprising: a patient sample inlet; a reference sample inlet; a gas analysis chamber; and a solenoid valve governing the supply of gas to said gas analysis chamber from said patient sample inlet and said reference sample inlet, said solenoid valve being operative for defining a normally-open passageway between said patient sample inlet and said gas analysis chamber and a normally-closed passageway between said reference sample inlet and said gas analysis chamber, said gas analyzer being characterized in that it has a rise time which does not exceed 10 milliseconds at a flow rate of 50 ml/min.

21. A gas analyzer comprising: a patient sample inlet; a reference sample inlet; a gas analysis chamber; and a solenoid valve governing the supply of gas to said gas analysis chamber from said patient sample inlet and said reference sample inlet and comprising a magnet, said solenoid valve being operative for defining a normally-open passageway between said patient sample inlet and said gas analysis chamber and a normally-closed-passageway between said reference sample inlet and said gas analysis chamber, wherein said passageway between said patient sample inlet and said gas analysis chamber is maintained open at least partially by a force applied by said magnet.

22. A gas analyzer according to claim 15 and wherein said passageway between said patient sample inlet and said gas analysis chamber has significantly less dead space than said passageway between said reference sample inlet and said gas analysis chamber.

23. A gas analyzer according to claim 15 and wherein said solenoid valve comprises a partially hollow plunger.

24. A gas analyzer according to claim 15 and wherein said solenoid valve comprises a push valve.

25. A gas analyzer according to claim 15 and wherein said solenoid valve comprises a magnet operative to maintain said passageway between said patient sample inlet and said gas analysis chamber open irrespective of the orientation of said solenoid valve, when said solenoid valve is not actuated.

26. A gas analyzer according to claim 15, said gas analyzer being characterized in that it has a rise time which does not exceed 50 milliseconds at a flow rate of 50 ml/min.

27. A gas analyzer according to claim 26, and wherein said rise time does not exceed 30 milliseconds at a flow rate of 50 ml/min.

28. A gas analyzer according to claim 26, and wherein said rise time does not exceed 10 milliseconds at a flow rate of 50 ml/min.
